(19) United States
(12) Patent Application Publication
(10) Pub. No.: US 2026/0335002 A1
(43) Pub. Date: Oct. 29, 2026

(54) Systems And Methods For Sizing An Electrode Probe In An Ablation Procedure

(71) Applicant: Stryker Corporation, Portage, MI (US)

(72) Inventor: Beau Michael Kidman, Kalamazoo, MI (US)

(73) Assignee: Stryker Corporation, Portage, MI (US)

(21) Appl. No.: 18/997,297

(22) PCT Filed: Jul. 21, 2023
(86) PCT No.: PCT/US2023/028321

§ 371 Date: Jan. 21, 2025

Related U.S. Application Data
(60) Provisional application No. 63/391,442, filed on Jul. 22, 2022.

(51) Int. Cl. A61B 18/14; A61B 18/00; A61B 90/00

(52) U.S. Cl. CPC A61B 18/148; A61B 90/08; A61B 2018/00339; A61B 2018/00577; A61B 2090/0807

(57) ABSTRACT

Systems and methods for ablating tissue within a vertebral body. A shaft of the drill is directed through an access cannula to form a tissue path. A first indicia or a second indicia on the shaft of the drill is determined to be in registration with a cannula hub of the access cannula. The first and second indicia may be different colors. The colors are correlated with complementary indicia associated with a first and second probe. Based on the correlation, the first probe or second probe is selected to be directed through the access cannula and into the formed tissue path to ablate the tissue. The indicia may be longitudinally positioned on the drill shaft, such as being inset along a side of the drill shaft. The indicia may be substantially coincident, and a length of the second indicia may be greater than a length of the first indicia.

### PRIORITY CLAIM

This application claims priority to and all the benefits of United States Provisional Patent Application No. 69/391,442, filed Jul. 22, 2022, the entire contents of which is hereby incorporated by reference.

### BACKGROUND

Radiofrequency (RF) energy is utilized to ablate diseased tissue such as sensory nerves, intraosseous nerves, or intraosseous tumors, among other anatomic structures. An electrode probe may be coupled to an electrosurgical console, and the RF energy is conducted from emitters of the electrode probe to adjacent tissue to create a lesion at the treatment location. Of particular interest is destruction of intraosseous tumors within a vertebral body in which the RF energy heats the tissue to destroy the cells of the tumor.

A transpedicular approach includes directing an access cannula through the pedicle of the vertebral body. The electrode probe may be directed through the access cannula to be positioned within or adjacent at least a portion of the tumor. It is desirable to ablate as much as of the tumor as possible, and therefore it is known to provide kits each with one of multiple “sizes” of electrode probes from which to choose, more particularly electrode probes having increasing lengths of emitters to generate an ablation zone that spans a greater width or length of the tumor. Regardless of the selected size of the electrode probe, a proximal end of the proximal emitter should not remain within the access cannula to avoid electrical shorting or other suboptimal ablation patterns. At the same time, owing to the fibrous tissue characteristics of the tumor itself and the relative fragility of the electrode probes, it is often indicated to bore or drill a tissue path through at least a portion of the tumor. If the tissue path is of insufficient length or if the incorrect electrode probe from the kit is selected for a given length of the tissue path, contact between the distal end of the electrode probe and an end of the tissue path may prevent the proximal emitter from emerging from within the access cannula.

It is known to provide indicia on a shaft of surgical instrumentation. In one example, the indicia are thin rings at fixed increments to correlate to a numerical depth by which a distal portion of the instrumentation is exposed beyond the access cannula. Such indicia do not intuitively provide the user with information regarding the corresponding size of the electrode probe to be selected. In other words, it would be preferable to not require the user to manually count exposed ring-type indicia to then calculate and determine the corresponding size of the electrode probe. For another example, U.S. Pat. No. 10,729,490 , issued Aug. 4, 2020, hereby incorporated by reference in its entirety, discloses a system in which ring-type indicia on a drill is aligned with the access cannula to provide an indication of a resulting size of the ablation zone generated with the corresponding electrode probe, also referred to therein as ablation zone mapping. Such a system, for it to provide its intended benefit, requires one of the thin rings to be precisely aligned with the access cannula, which may correlate to a suboptimal depth of the tissue path formed by the drill. Alternatively, the optimal depth formed by the drill (e.g., as desired by the user) may result in misalignment of the ring-type indicia in which instance the ablation zone mapping may be imprecise.

Therefore, there is a need in the art for improved systems and methods to provide for intuitive selection of the size of electrode probe based on the user selected depth of the tissue path formed by the drill. It further desirable to provide for readily discernable information as to a position of the emitters within the formed tissue path.

### BRIEF DESCRIPTION OF THE DRAWINGS

FIG. 1 is a view of a vertebrae including a pedicle through which an access cannula is directed. A drill is being directed through the access cannula to form a tissue path through an intraosseous tumor.

FIG. 2A is an elevation view of an ablation system that includes the access cannula, the drill, and two electrode probes of different sizes.

FIG. 2B is an elevation view of an ablation system that includes the access cannula, the drill, and four electrode probes of different sizes.

FIG. 3 shows the system in an arrangement in which the tissue path is formed by the drill, and wherein a distal margin of a first indicia is aligned with the access cannula. A first probe is positionable within the tissue path for an end of the first probe to be adjacent to an end of the tissue path.

FIG. 4 shows the system in another arrangement in which the tissue path is formed by the drill, and wherein a proximal margin of the first indicia is aligned with the access cannula. The first probe is positionable within the tissue path for the end of the first probe to be spaced apart from the end of the tissue path.

FIG. 5 shows the system in another arrangement in which the tissue path is formed by the drill, and wherein a distal margin of a second indicia is aligned with the access cannula. A second probe is positionable within the tissue path for the end of the second probe to be adjacent to the end of the tissue path.

FIG. 6 shows the system in another arrangement in which the tissue path is formed by the drill, and wherein a proximal margin of the second indicia is aligned with the access cannula. The second probe is positionable within the tissue path for the end of the second probe to be spaced apart from the end of the tissue path.

FIG. 7A is an elevation view of the drill of FIG. 2.

FIG. 7B is a detailed view of the indicia of the drill within circle 7B.

FIG. 7C is a is a detailed view of another implementation of the drill in which there are four indicia to correspond to one of four electrode probes of different sizes.

FIG. 8 is an elevation view of another implementation of the drill in which the indicia are opaque bands.

FIG. 9 is an elevation view of another implementation of the drill in which the indicia are translucent bands.

### DETAILED DESCRIPTION

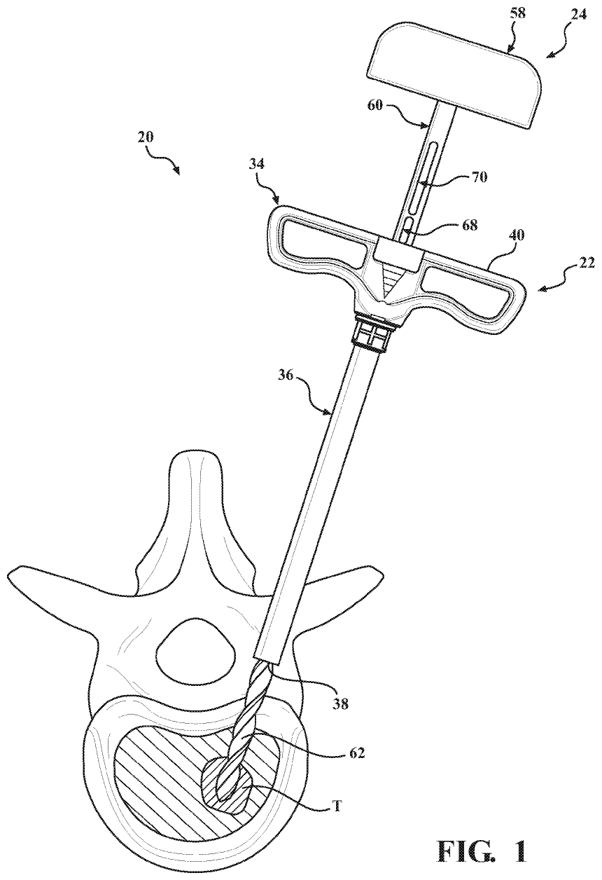

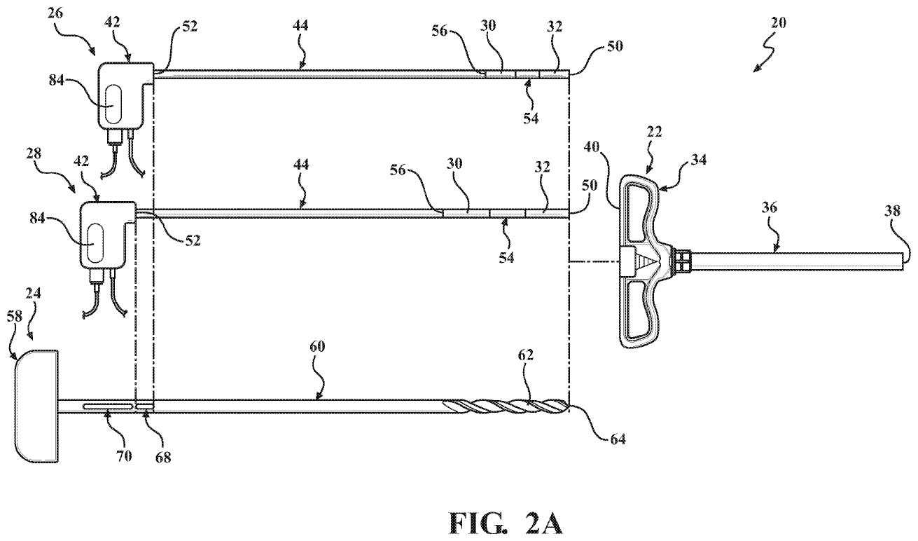

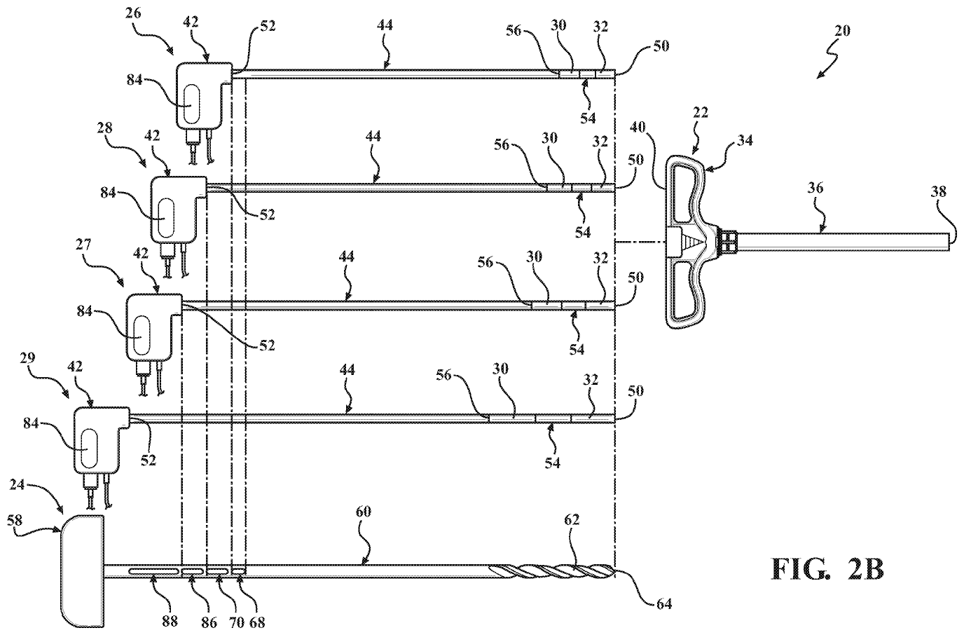

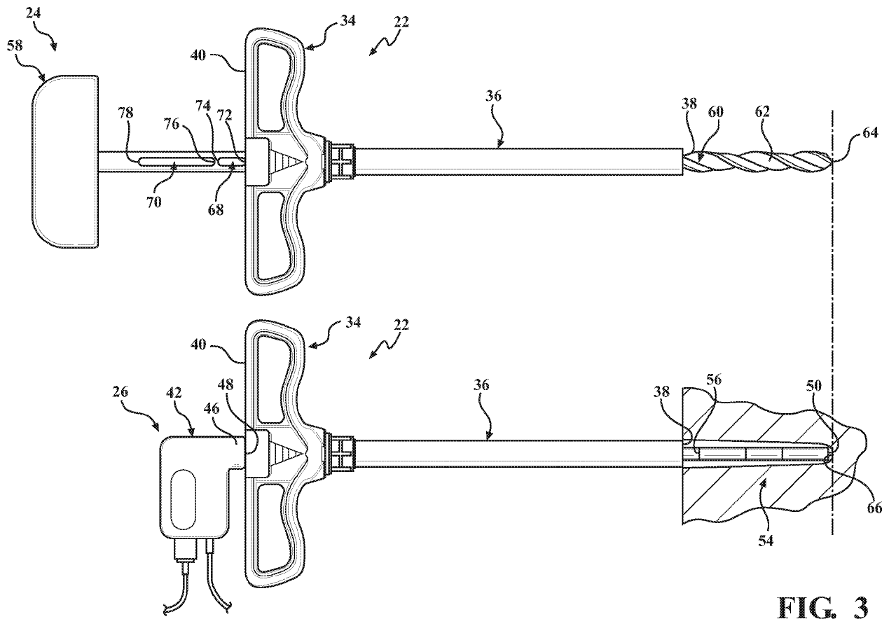

The present disclosure is directed to an ablation system 20 in which an electrode probe is efficiently and intuitively “sized” based on a depth of a tissue path formed with a drill 24 within tissue, for example, through a tumor (T) within a vertebral body. Referring to FIGS. 1 and 2, the ablation system 20 includes an access cannula 22, the drill 24, a first probe 26, and a second probe 28. The ablation system 20 further includes a electrosurgical console (not shown) to which the first and second probes 26, 28 are configured to be removably coupled to generate and transmit the RF energy. An exemplary console is disclosed in commonly-owned International Publication No. WO 2023/009697, published Feb. 2, 2023, the entire contents of which are hereby incorporated by reference. As to be further described, the access cannula 22 is configured to be percutaneously directed through the pedicle of the vertebral body, after which the drill 24 is directed through the access cannula 22 to form the tissue path. FIG. 1 shows the drill 24 further forming the tissue path through the tumor (T) within the vertebral body. The drill 24 is removed from the access cannula 22, and at least one of the first and second probes 26, 28 is directed through the access cannula for proximal and distal emitters 30, 32 to be positioned within the tissue path (see FIGS. 3-6). The electrosurgical console is operated to transmit the RF energy through the emitters 30, 32 to be delivered into and destroy cells of the tumor.

The access cannula 22 includes a cannula hub 34, and a cannula shaft 36 extending distally from the cannula hub 34 to provide a distal end 38 of the access cannula 22. A trocar (not shown) may be removably disposed within the access cannula 22 and include a sharp tip configured to pierce cortical bone during percutaneous insertion of the access cannula 22. The cannula hub 34 includes a proximal surface 40 that provides a visual and structural datum to be described in further detail. It is understood that the access cannula 22 may include a Luer fitting (not shown) coupled to the cannula hub 34 through which the drill 24 and the first and second probes 26, 28 are configured to be directed. In such an arrangement, a proximal edge of the Luer fitting may provide the visual and structural datum.

The first and second probes 26, 28 each include a probe hub 42, and a probe shaft 44 extending from the probe hub 42. The probe hub 42 provides a handle for the user to grasp a respective one of the first and second probes 26, 28. The probe hub 42 may include a hub neck 46 defining a distal surface 48 of the probe hub 42, wherein the probe shaft 44 extends from the hub neck 46. The proximal and distal emitters 30, 32 are disposed on the probe shaft 44. The distal emitter 32 may be spaced apart from the proximal emitter 30 by a portion of the probe shaft 44 such that the first and second probes 26, 28 are self-grounding bipolar probes. The distal emitter 32 may define a distal end 50 of the first and second probes 26, 28. Further details of the probes are disclosed in commonly-owned International Publication No. WO2020/198150, published Nov. 5, 2020, the entire contents of which are hereby incorporated by reference. The first and second probes 26, 28 are different sizes. In particular, FIGS. 2A and 2B show that the first probe 26 may be considered smaller than the second probe 28. The probe shaft 44 may include a length defined between the distal end 50 and a proximal end 52 at an interface with the probe hub 42. The length of the probe shaft 44 of the first probe 26 is less than the length of the probe shaft 44 of the second probe 28. It is further understood that an active tip region 54 of the first probe 26—defined between the distal end 50 and a proximal end 56 of the proximal emitter 30—is less than a counterpart active tip region 54 of the second probe 28. In other words, the proximal and distal emitters 30, 32 of the second probe 28 may be larger.

Referring now to FIGS. 1, 2A, 2B, 7A and 7B, the drill 24 includes a drill hub 58, and a drill shaft 60 extending distally from the drill hub 58. The drill shaft 60 includes cutting geometries 62 at or near a distal end 64 of the drill shaft 60. A length of the drill shaft 60 is greater than the length of the probe shaft 44 of the first probe 26 and the second probe 28. As mentioned, the drill 24 is configured to be directed coaxially through the access cannula 22, and the drill hub 58 may be manipulated to cause the cutting geometries 62 to form the tissue path. FIGS. 3-6 show representations of the tissue path in which the path may be formed as a channel having an end 66. Once an initial tissue path is formed, an exemplary workflow may include confirming the distal end 64 of the drill shaft 60 on imaging, for example, intraoperative fluoroscopy. For example, the end 66 of the tissue path may be determined by visualizing the distal end 64 of the drill shaft 60. It is often indicated to drill to or beyond a distal margin of the tumor such that the ablation zone of the subsequently positioned first or second probe 26, 28 includes at least most or an entirety of the tumor.

First and second indicia 68, 70 are positioned on a proximal portion of the drill shaft 60. The first indicia 68 is positioned distal to the second indicia 70. The first indicia 68 may include a distal margin 72 and a proximal margin 74 with a length defined therebetween. Likewise, the second indicia 70 may include a distal margin 76 and a proximal margin 78 with a length defined therebetween. The length of the second indicia 70 is greater than a length of the first indicia 68. In alternative configurations, the length of the first indicia 68 is greater, or the respective lengths may the same. The length of the first indicia 68 may be within the range of about 0.3 to 1.0 centimeters (cm), and more particularly within the range of about 0.4 to 0.6 cm. The length of the second indicia 70 may be within the range of about 0.5 to 2.0 centimeters (cm), and more particularly within the range of about 0.75 to 1.25 cm. The length of the first indicia 68 may be equal to a difference in lengths between the probe shafts 44 of the first and second probes 26, 28. For example, the active tip region 54 of the first probe 26 and the second probe 28 may be fifteen and twenty millimeters, respectively, and therefore the length of the first indicia 68 may be approximately five millimeters. The respective lengths are at least sufficient for the first and second indicia 68, 70 to be aligned with the proximal surface 40 of the cannula hub 34 over a range of positions of the drill 24 relative to the access cannula 22. In other words, it should be understood that each of the first and second indicia 68, 70 are not thin rings with negligible length, which would otherwise become obscured within the cannula hub 34 with only minimal distal movement of the drill 24 relative to the access cannula 22. In the exemplary implementation, there are exactly two indicia to correspond to the first and second probes 26, 28, however, more or less indicia may be provided.

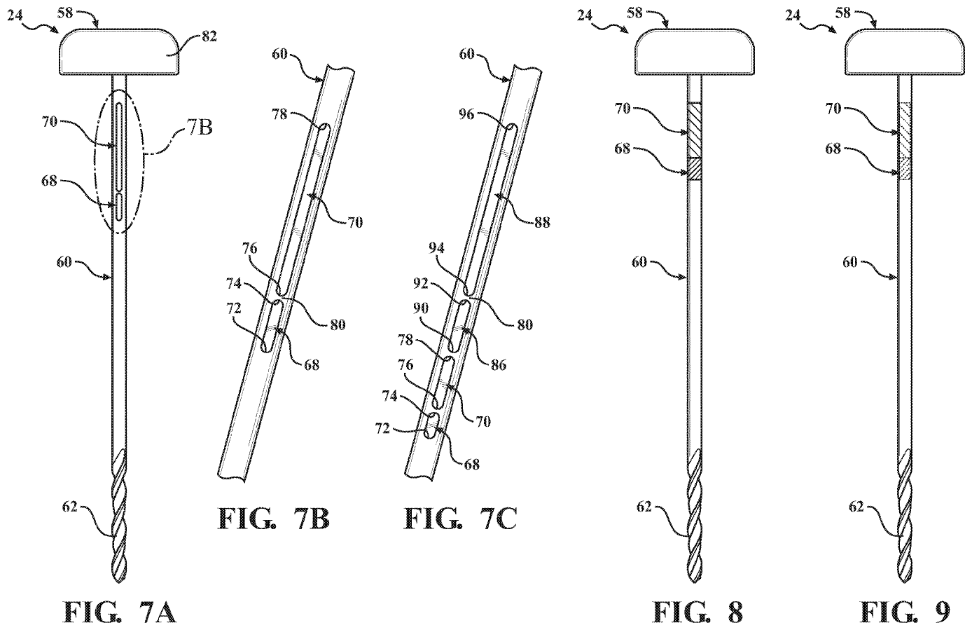

The distal margin 76 of the second indicia 70 may be substantially coincident with the proximal margin 74 of the first indicia 68. In other words, the first indicia 68 and the second indicia 70 may be abutting or adjacent to one another such that there is little to no distance between the same. As best shown in FIGS. 7A and 7B, only a thin portion 80 of the drill shaft 60 is situated between the first indicia 68 and the second indicia 70. The thin portion 80 may be less than one millimeter, or may not be present whatsoever. For example, FIGS. 8 and 9 show the first indicia 68 and the second indicia 70 abutting one another.

The first and second indicia 68, 70 may be disposed on or formed into the proximal portion of the drill shaft 60 in any number of suitable manners. FIGS. 7A and 7B show the first indicia 68 and the second indicia 70 as being elongate, linear, and longitudinally oriented along a side of the proximal portion of the drill shaft. For example, the first and second indicia 68, 70 may be inset within the drill shaft 60 and formed by a milling operation, a laser operation, or the like. In one variant, the first and second indicia 68, 70 are elongate, linear, and longitudinally inset along opposing sides of the proximal portion of the drill shaft 60. The opposing sides may be defined by being aligned with primary faces 82 (one shown) of the drill hub 58. FIG. 8 shows the first indicia 68 and the second indicia 70 being opaque bands extending around the drill shaft 60, and FIG. 9 shows the first indicia 68 and the second indicia 70 being translucent bands extending around the drill shaft 60. In still another example, the bands may be colored adhesive applied to the drill shaft 60. The bands, as used herein, are intended to indicate a length greater than that of thin rings with negligible length.

The first indicia 68 may be of a first color and the second indicia 70 of second color that is different than the first color. For example, the first color is blue and the second color is yellow, but other color combinations are contemplated. Likewise, other types of indicia may be utilized such as texturizing, ridges, shapes, or other visually-discernable patterns that are not feasible for thin ring-type indicia that have negligible length. The probe hub 42 of each of the first and second probes 26, 28 may include an indicia 84 or label (see FIGS. 2A and 2B) with the indicia 84 of the first probe 26 corresponding to the first indicia 68 and the indicia 84 of the second probe 28 corresponding to the second indicia 70. For example, the indicia 84 may include text of a corresponding color. Owing to the corresponding colors, the first and second indicia 68, 70 intuitively and quickly provide information to the user as to which of the first and second probes 26, 28 should be used without concern for the tissue path being of insufficient depth. In other words, the user may readily ascertain, based on the first and second indicia 68, 70 being aligned with the visual datum provided by the proximal surface 40 of the cannula hub 34 (or the Luer fitting), which of the first and second probes 26, 28 is sized to the tissue path formed by the drill 24. The alignment may also provide information to or an awareness by the user as to whether the probe 26, 28 will be positioned adjacent to or spaced apart from the end 66 of the tissue path. The first and second indicia 68, 70 may not necessarily provide any information as to the size of the ablation zone or ablation zone mapping.

As shown in FIG. 2A, a first distance between the distal margin 72 of the first indicia 68 and the distal end 64 of the drill shaft 60 is configured to be equal to a first probe length defined between the probe hub 42 of the first probe 26 and the distal end 50 of the probe shaft 44. Likewise, a second distance between the proximal margin 74 of the first indicia 68 and the distal end 64 of the drill shaft 60 is configured to be equal to a second probe length defined between the probe hub 42 of the second probe 28 and the distal end 50 of the probe shaft 44. Consequently, if the tissue path is formed with the first indicia 68 being visible adjacent the cannula hub 34, the formed tissue path may be of insufficient path length to permit the active tip region 54 of the second probe 28 to be positioned beyond the distal end 38 of the access cannula 22.

Exemplary methods may therefore include directing the distal end 64 of the drill shaft 60 beyond the distal end 38 of the access cannula 22 to form the tissue path of a desired length. The distal end 64 of the drill shaft 60 may be confirmed on the imaging, and adjusted accordingly if desired. With the drill 24 in the desired position, the drill hub 58 is spaced from the cannula hub 34 and the proximal portion of the drill shaft 60 is exposed. The method includes determining whether the first indicia 68 or the second indicia 70 is in registration with the cannula hub 34 of the access cannula 22. Owing to the contrasting colors of the first and second indicia 68, 70, for example, the user may quickly ascertain the information. FIG. 3 shows the first indicia 68 aligned with the proximal surface 40 of the access cannula 22. The drill 24 may be removed from the access cannula 22.

The method includes correlating one of the first and second indicia 68, 70 that is in registration with the cannula hub 34 of the access cannula 22 with the complementary indicia 84 associated with a respective one of the first probe 26 and the second probe 28. In the exemplary implementation, the first color or the second color in registration with the cannula hub 34 of the access cannula 22 is correlated with a corresponding color of complementary indicia 84. One of the first and second probes 26, 28 is selected based on the corresponding color. In other words, if the first indicia 68 aligned with the cannula hub 34, it can be assumed that the path length is at least sufficient to accommodate the active tip region 54 of the first probe 26, and if the second indicia 70 aligned with the cannula hub 34, it can be assumed that the path length is at least sufficient to accommodate the active tip region 54 of the second probe 28. The method includes directing the selected one of the first probe 26 and the second probe 28 through the access cannula 22 and into the formed tissue path. The distal surface 48 of the probe hub 42, for example, may be positioned to abut the proximal surface 40 of the cannula hub 34 (or Luer fitting) providing the structural datum. Owing to the relative lengths of the probe shaft 44 and the cannula shaft 36, it can be further assumed that the active tip region 54, including the proximal end 56 of the proximal emitter 30, is exposed beyond the distal end 38 of the access cannula 22. The selected one of the first and second probe 26, 28 is operated (with the RF energy provided by the console) to ablate the tissue adjacent the formed tissue path.

The intuitiveness and efficiency of the ablation system 20 of the present disclosure are readily appreciable. Stated simply, should any portion of the first indicia 68 be visible, the user understands to not use the second probe 28. Otherwise, the proximal emitter 30 of the second probe 28 may not be exposed beyond the access cannula 22 prior to the distal end 50 of the second probe 28 bottoming out within the tissue path. Likewise, should only the second indicia 70 be visible, the user understands to not use the first probe 26, as it will be too short to meaningfully occupy the formed tissue path. Should the user still wish to use the first probe 26 in such an instance, it may be indicated to reposition the access cannula 22 in order to reach the target location. In instances where the first and second probes 26, 28 are packaged separately in sterile kits, the user may advantageously avoid unpackaging the unselected probe. The packaged, sterile probe may be saved for a subsequent ablation procedure, thereby reducing cost and waste.

Further information may be realized based on a position of the margins 72, 74, 76, 78 relative to the cannula hub 34 of the access cannula 22. In instances where the user may visualize a portion of the drill shaft 60 distal to the distal margin 72 of the first indicia 68, the user would appreciate that the first probe 26 (and the second probe 28) is too long for the formed tissue path. The user would understand to further advance the drill 24 relative to the access cannula 22 to deepen the tissue path (e.g., at least until the distal margin 72 of the first indicia 68 is aligned with the proximal surface 40). FIG. 3 shows the distal margin 72 of the first indicia 68 aligned with the proximal surface 40 of the access cannula 22. The arrangement is indicative that the path length is approximately equal to the distance by which the first probe 26 is configured to extend beyond the distal end 38 of the access cannula 22. The distal end 50 of the first probe 26 is adjacent to or abutting the end 66 of the tissue path.

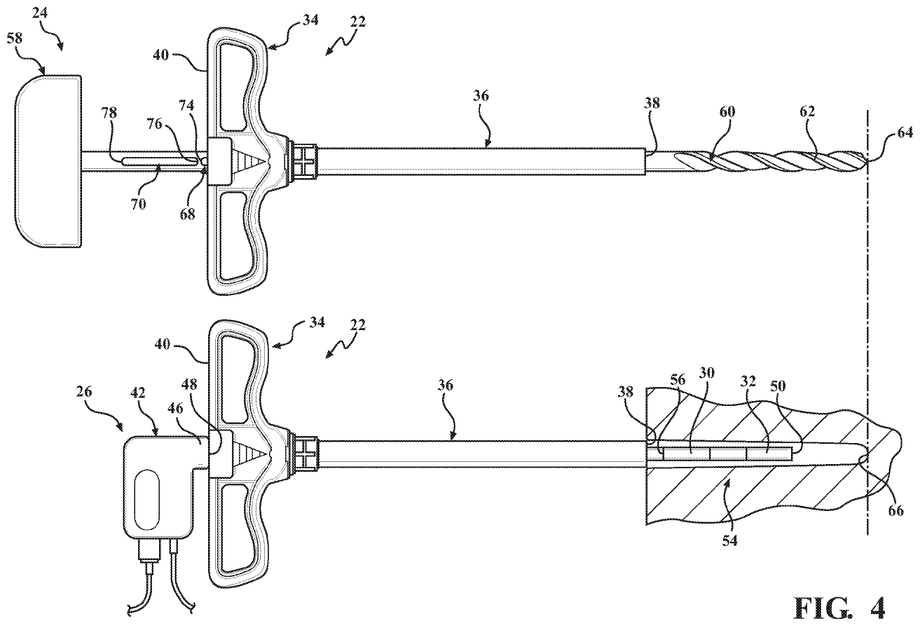

In certain methods, the proximal margin 74 of the first indicia 68 may be correlated as being in registration with the cannula hub 34. FIG. 4 shows the proximal margin 74 of the first indicia 68 visible adjacent to the proximal surface 40 of the access cannula 22. The user may select the first probe 26 based on the correlation, and direct the first probe 26 through the access cannula 22 and into the formed tissue path. In such an arrangement, the user would appreciate that the distal end 50 of the first probe 26 is to be spaced apart from the end 66 of the tissue path with the probe hub 42 engaging the cannula hub 34. FIG. 4 shows an unoccupied portion of the tissue path. Based on a proportion by which the first indicia 68 is visible proximal to the cannula hub 34, the user can intuitively deduce a size of the gap to be within the tissue path distal to the distal end 50 of the first probe 26. In other instances, the proximal margin 74 of the first indicia 68 may be aligned with or just internal to the cannula hub 34 such that the first indicia 68 is not visible, or alternatively the thin portion 80 between the first and second indicia 68, 70 may be in registration with the cannula hub 34. Since the user cannot see the first indicia 68, the user may select the second probe 28 based on the correlation, and the second probe 28 may be directed through the access cannula 22 and into the formed tissue path.

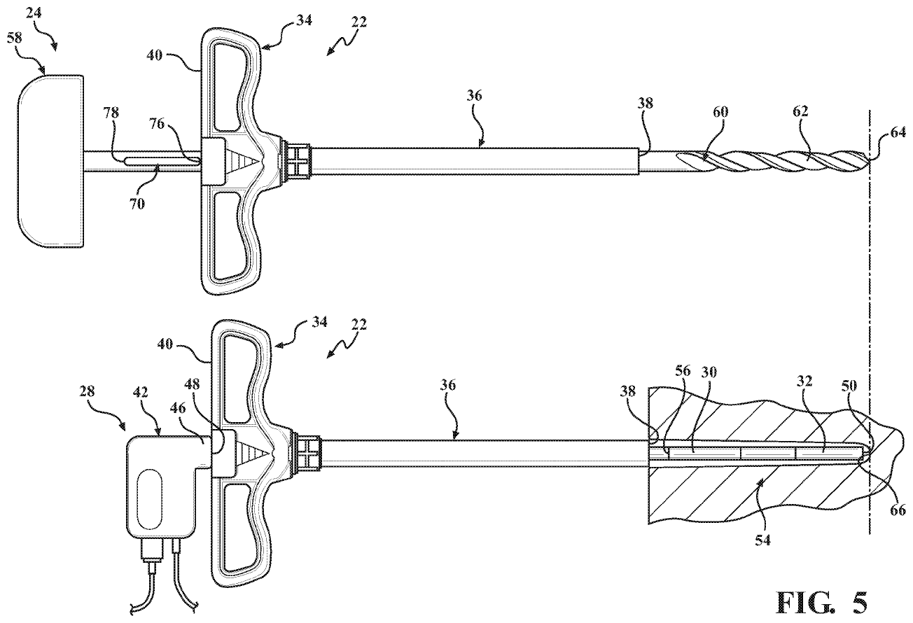

In certain methods, the distal margin 76 of the second indicia 70 may be correlated as being in registration with the cannula hub 34. FIG. 5 shows the distal margin 76 of the second indicia 70 aligned with the proximal surface 40 of the access cannula 22. The user may select the second probe 28 based on the correlation, and the second probe 28 may be directed through the access cannula 22 and into the formed tissue path. The distal end 50 of the second probe 28 is near, adjacent to, or abutting the end 66 of the formed tissue path with the probe hub 42 engaging the cannula hub 34. A minimal gap may be present corresponding to a distance between the proximal margin 74 of the first indicia 68 and the distal margin 76 of the second indicia 70.

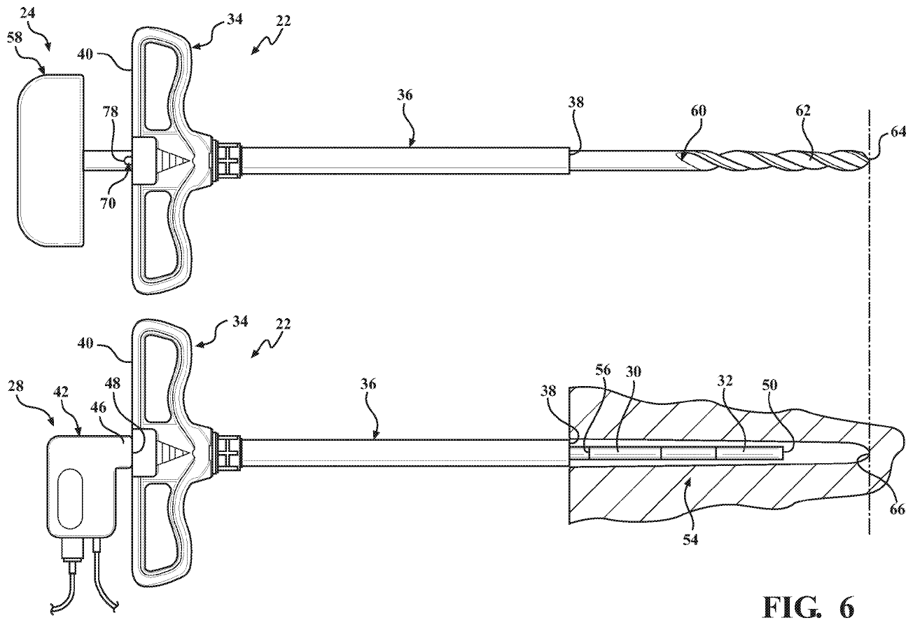

In certain methods, the proximal margin 78 of the second indicia 70 may be correlated as being in registration with the cannula hub 34. FIG. 6 shows the proximal margin 78 of the second indicia 70 aligned with the proximal surface 40 of the access cannula 22. The user may select the second probe 28 based on the correlation, and the second probe 28 may be directed through the access cannula 22 and into the formed tissue path. The distal end 50 of the second probe 28 is spaced apart from the end 66 of the tissue path with the probe hub 42 engaging the cannula hub 34. FIG. 6 shows another unoccupied portion of the tissue path. Based on a proportion by which the second indicia 70 is visible proximal to the cannula hub 34, the user can intuitively deduce the size of the gap to be within the tissue path distal to the distal end 50 of the second probe 28.

The objects of the present disclosure may be extended to systems including three, four, or five or more probes. For example, FIGS. 2B and 7C are representative of the ablation system 20 in which the drill shaft 60 includes first, second, third, and fourth indicia 68, 70, 86, 88 respectively corresponding to the first probe 26, the second probe 28, a third probe 27, and a fourth probe 29. The third and fourth probes 27, 29 and the third and fourth indicia 86, 88 may correspond to the first and second probes 26, 28 and the first and second indicia 68, 70, respectively. In other words, the active tip region 54 of the third probe 27 and the fourth probe 29 may be fifteen and twenty millimeters, respectively, and therefore the length of the third indicia 86 may be approximately five millimeters.

In the present implementation, the active tip region 54 of the first probe 26 and the second probe 28 may be seven and ten millimeters, respectively, and therefore the length of the first indicia 68 and the second indicia 70 may be approximately three and five millimeters, respectively. More particularly, the first indicia 68 may include the distal margin 72 and the proximal margin 74 with the length defined therebetween, and the second indicia 70 may include the distal margin 76 and the proximal margin 78 with the length defined therebetween. The length of the second indicia 70 is greater than a length of the first indicia 68. The length of the first indicia 68 may be equal to a difference in lengths between the probe shafts 44 of the first and second probes 26, 28. Further in the present implementation, the active tip region 54 of the third probe 27 and the fourth probe 29 may be fifteen and twenty millimeters, respectively, and therefore the length of the third indicia 86 may be approximately five millimeters. The third indicia 86 may include a distal margin 90 and a proximal margin 92 with a length defined therebetween, and the fourth indicia 88 may include a distal margin 94 and a proximal margin 96 with a length defined therebetween. The length of the second indicia 86 may be equal to a difference in lengths between the probe shafts 44 of the second and third probes 27, 28. The length of the third indicia 86 may be equal to a difference in lengths between the probe shafts 44 of the third and fourth probes 26, 28. Therefore, the length of the third indicia 86 may be the same as the length of the second indicia 70.

The first, second, third and fourth indicia 68, 70, 86, 88 may be elongate, linear, and longitudinally oriented along a side of the proximal portion of the drill shaft. For example, the first, second, third and fourth indicia 68, 70, 86, 88 may be inset within the drill shaft 60 and formed by a milling operation, a laser operation, or the like. The first, second, third and fourth indicia 68, 70, 86, 88 may be different colors. For example, the first color is blue and the second color is pink, the third color is green, and the fourth color is yellow, but other color combinations are contemplated. The probe hub 42 of each of the first, second, third and fourth probes 26, 27, 28, 29 may include an indicia 84 or label (see FIGS. 2A and 2B) with corresponding indicia 84. Owing to the corresponding colors, the first, second, third and fourth indicia 68, 70, 86, 88 intuitively and quickly provide information to the user as to which of the first, second, third and fourth probes 26, 27, 28, 29should be used without concern for the tissue path being of insufficient depth.

Exemplary methods may therefore include directing the distal end 64 of the drill shaft 60 beyond the distal end 38 of the access cannula 22 to form the tissue path of a desired length. The distal end 64 of the drill shaft 60 may be confirmed on the imaging, and adjusted accordingly if desired. With the drill 24 in the desired position, the drill hub 58 is spaced from the cannula hub 34 and the proximal portion of the drill shaft 60 is exposed. The method includes determining whether the first, second, third and fourth indicia 68, 70, 86, 88 is in registration with the cannula hub 34 of the access cannula 22. Owing to the contrasting colors of the first, second, third and fourth indicia 68, 70, 86, 88, for example, the user may quickly ascertain the information.

The method includes correlating one of the first, second, third and fourth indicia 68, 70, 86, 88 that is in registration with the cannula hub 34 of the access cannula 22 with the complementary indicia 84 associated with a respective one of the first, second, third and fourth probes 26, 27, 28, 29. One of the first, second, third and fourth probes 26, 27, 28, 29 is selected based on the corresponding color. The selected one of the first, second, third and fourth probes 26, 27, 28, 29 is directed through the access cannula 22 and into the formed tissue path. Owing to the relative lengths of the probe shaft 44 and the cannula shaft 36, it can be further assumed that the active tip region 54, including the proximal end 56 of the proximal emitter 30, is exposed beyond the distal end 38 of the access cannula 22. The selected one of the first, second, third and fourth probes 26, 27, 28, 29 is operated to ablate the tissue adjacent the formed tissue path.

During deployment of the drill 24, should any portion of the first indicia 68 be visible, the user understands to not use the second, third or fourth probes 27, 28, 29. Should any portion of the second indicia 70 be visible, the user understands to not use the third or fourth probes 27, 29. Should any portion of the third indicia 86 be visible, the user understands to not use the fourth probe 29. Finally, should only the fourth indicia 88 be visible, the user understands to not use the first, second, or third probes 26, 27, 28, as it will be too short to meaningfully occupy the formed tissue path. Further aspects of the method of the ablation system 20 including the four probes are similar to those previously described for the two-probe system, and are hereby incorporated by reference. As mentioned, the ablation system 20 may be modified to accommodate any number of probes.

The foregoing disclosure is not intended to be exhaustive or limit the invention to any particular form. The terminology which has been used is intended to be in the nature of words of description rather than of limitation. Many modifications and variations are possible in light of the above teachings and the invention may be practiced otherwise than as specifically described. It should be understood that treatment parameters other than impedance and temperature may be utilized with the aforementioned techniques. Furthermore, it is contemplated that the treatment parameters may be controlled or defined in manners different from the techniques described. It is further understood that the objects of the present disclosure may be used with microwave energy or electrical energy other than radiofrequency energy.

1. A method of ablating tissue within a vertebral body with a system including an access cannula including a cannula hub, a drill including a drill hub, and a first probe and a second probe of different lengths and each including a probe hub, the method comprising:
providing access to within the vertebral body with the access cannula;
directing a distal end of a shaft of the drill beyond a distal end of the access cannula to form a first tissue path of a first path length or a second tissue path of a second path length greater than the first path length, wherein the drill hub is spaced from the cannula hub and a proximal portion of the drill shaft is exposed;
determining whether a first indicia of a first color or a second indicia of a second color is in registration with the cannula hub of the access cannula, wherein the first indicia and the second indicia are longitudinally sized and positioned on the proximal portion of the drill shaft to be associated with a respective one of the first path length and the second path length for receiving a respective one of the first probe and the second probe;
correlating one of the first color and the second color that is in registration with the cannula hub of the access cannula with a corresponding color of complementary indicia associated with a respective one of the first probe and the second probe;
selecting one of the first probe and the second probe based on the correlated one of the first color and the second color;
removing the drill from the access cannula;
directing the selected one of the first probe and the second probe through the access cannula and into the formed first tissue path or the formed second tissue path; and
operating the selected one of the first probe and the second probe to ablate the tissue adjacent the formed first tissue path or the formed second tissue path.

2. The method of claim 1, further comprising:
correlating a proximal margin of the first indicia as being in registration with the cannula hub with the first indicia partially visible;
selecting the first probe; and
directing the first probe through the access cannula and into the formed first tissue path such that the distal end of the first probe is spaced apart from an end of the first tissue path with the first probe hub engaging the cannula hub.

3. The method of claim 1, further comprising:
correlating a proximal margin of the first indicia as being in registration with the cannula hub with the first indicia not visible;
selecting the second probe; and
directing the second probe through the access cannula and into the formed second tissue path such that the distal end of the second probe is at an end of the second tissue path with a second probe hub engaging the cannula hub.

4. The method of claim 1, further comprising:
correlating a thin portion of the drill shaft between the first indicia and the second indicia as being in registration with the cannula hub with the first indicia not visible;
selecting the second probe; and
directing the second probe through the access cannula and into the formed second tissue path.

5. The method of claim 1, further comprising:
correlating a distal margin of the second indicia as being in registration with the cannula hub;
selecting the second probe; and
directing the second probe through the access cannula and into the formed second tissue path.

6. The method of claim 1, further comprising:
correlating a proximal margin of the second indicia as being in registration with the cannula hub;
selecting the second probe; and
directing the second probe through the access cannula and into the formed second tissue path such that the distal end of the second probe is spaced apart from the end of the second tissue path with the second probe hub engaging the cannula hub.

7. A method of ablating tissue within a vertebral body with a system including an access cannula including a cannula hub, a drill including a drill hub, and a first probe and a second probe of different lengths and each including a probe hub, the method comprising:
providing access to within the vertebral body with the access cannula;
directing a distal end of a shaft of the drill beyond a distal end of the access cannula to form a first tissue path of a first path length or a second tissue path of a second path length greater than the first path length, wherein the drill hub is spaced from the cannula hub and a proximal portion of the drill shaft is exposed;
determining whether a first indicia of a first color or a second indicia of a second color on the proximal portion of the drill shaft is in registration with the cannula hub of the access cannula;
correlating one of the first color or the second color with a corresponding color of complementary indicia associated with a respective one of the first probe and the second probe, wherein a distal margin of the first indicia being in registration with the cannula hub provides for a proximal emitter of the first probe to be exposed beyond the distal end of the access cannula and a distal end of the first probe to be positioned at an end of the first tissue path with a first probe hub engaging the cannula hub, and wherein the second probe has a length such that a proximal emitter of the second probe would not be exposed beyond the distal end of the access cannula based on a distal end of the second probe engaging the tissue at the end of the first tissue path;
selecting one of the first probe and the second probe based on the correlated one of the first color and the second color;
removing the drill from the access cannula;
directing the selected one of the first probe and the second probe through the access cannula and into the formed first tissue path or the formed second tissue path; and
operating the selected one of the first probe and the second probe to ablate the tissue adjacent the formed first tissue path or the formed second tissue path.

8. The method of claim 7, wherein a proximal margin of the first indicia being in registration with the cannula hub provides for the distal end of the first probe being spaced apart from the end of the first tissue path with the first probe hub engaging the cannula hub, wherein a distal margin of the second indicia being in registration with the cannula hub provides for the distal end of the second probe being at an end of the second tissue path with a second probe hub engaging the cannula hub, and wherein a proximal margin of the second indicia being in registration with the cannula hub provides for the distal end of the second probe being spaced apart from the end of the second tissue path with the second probe hub engaging the cannula hub.

9. The method of claim 7, further comprising confirming, on imaging, a position of the distal end of the drill shaft, wherein the step of determining whether the first indicia of the first color or the second indicia of the second color is in registration with the cannula hub is performed after the step of confirming the distal end of the drill shaft on the imaging.

10. The method of claim 7, wherein the first probe is packaged in a first probe kit and the second probe is packaged in a second probe kit, the method further comprising opening a singular one of the first probe kit and the second probe kit based on the correlated one of the first color and the second color such that the second probe kit remains usable in a subsequent ablation procedure.

11-23. (canceled)

24. The method of claim 1, wherein the step of determining whether the first indicia of the first color or the second indicia of the second color is in registration with the cannula hub is performed after the step of directing the distal end of the shaft of the drill beyond the distal end of the access cannula to form the first tissue path or the second tissue path.

25. The method of claim 1, further comprising confirming, on imaging, a position of the distal end of the drill shaft, wherein the step of determining whether the first indicia of the first color or the second indicia of the second color is in registration with the cannula hub is performed after the step of confirming the distal end of the drill shaft on the imaging.

26. The method of claim 1, wherein the first probe is packaged in a first probe kit and the second probe is packaged in a second probe kit, the method further comprising opening a singular one of the first probe kit and the second probe kit based on the correlated one of the first color and the second color such that the second probe kit remains usable in a subsequent ablation procedure.

27. The method of claim 1, wherein the first indicia and the second indicia are elongate, linear, and longitudinally arranged along a side of the proximal portion of the drill shaft.

28. The method of claim 7, wherein the first indicia and the second indicia are elongate, linear, and longitudinally arranged along a side of the proximal portion of the drill shaft.

29. A method of ablating tissue within a vertebral body with a system including an access cannula including a cannula hub, a drill including a drill hub, and a first probe and a second probe of different lengths and each including a probe hub, the method comprising:
providing access to within the vertebral body with the access cannula;
directing a distal end of a shaft of the drill beyond a distal end of the access cannula to form a first tissue path of a first path length or a second tissue path of a second path length greater than the first path length, wherein the drill hub is spaced from the cannula hub and a proximal portion of the drill shaft is exposed;
confirming, on imaging, a position of the distal end of the drill shaft;
thereafter, determining whether a first indicia of a first color or a second indicia of a second color is in registration with the cannula hub of the access cannula, wherein the first indicia and the second indicia are longitudinally sized and positioned on the proximal portion of the drill shaft to be associated with a respective one of the first path length and the second path length for receiving a respective one of the first probe and the second probe;
selecting one of the first probe and the second probe based on a correlated one of the first color and the second color;
removing the drill from the access cannula;
directing the selected one of the first probe and the second probe through the access cannula and into the formed first tissue path or the formed second tissue path; and
operating the selected one of the first probe and the second probe to ablate the tissue adjacent the formed first tissue path or the formed second tissue path.

30. The method of claim 29, further comprising:
correlating a proximal margin of the first indicia as being in registration with the cannula hub with the first indicia not visible;
selecting the second probe; and
directing the second probe through the access cannula and into the formed second tissue path such that the distal end of the second probe is at an end of the second tissue path with a second probe hub engaging the cannula hub.

31. The method of claim 29, further comprising:
correlating a thin portion of the drill shaft between the first indicia and the second indicia as being in registration with the cannula hub with the first indicia not visible;
selecting the second probe; and
directing the second probe through the access cannula and into the formed second tissue path.

32. The method of claim 29, further comprising:
correlating a distal margin of the second indicia as being in registration with the cannula hub;
selecting the second probe; and
directing the second probe through the access cannula and into the formed second tissue path.

33. The method of claim 29, further comprising:
correlating a proximal margin of the second indicia as being in registration with the cannula hub;
selecting the second probe; and
directing the second probe through the access cannula and into the formed second tissue path such that the distal end of the second probe is spaced apart from the end of the second tissue path with the second probe hub engaging the cannula hub.
